(19) United States
(12) Patent Application Publication
(10) Pub. No.: US 2024/0021316 A1
(43) Pub. Date: Jan. 18, 2024

(54) ERGONOMICS AID FOR COMPUTING ECOSYSTEMS

(71) Applicant: Dell Products L.P., Round Rock, TX (US)

(72) Inventors: Uboho Victor, San Antonio, TX (US); Tyler Ryan Cox, Austin, TX (US); Erik Summa, Austin, TX (US); Jake Mitchell Leland, Round Rock, TX (US); Marc Randall Hammons, Round Rock, TX (US); Ryan Nicholas Corner, Round Rock, TX (US)

(73) Assignee: Dell Products L.P., Round Rock, TX (US)

(21) Appl. No.: 17/812,043

(22) Filed: Jul. 12, 2022

(51) Int. Cl. G16H 50/30

(52) U.S. Cl. CPC G16H 50/30

(57) ABSTRACT

A data-driven, intelligent ergonomic behavioral aid based on uniquely captured telemetry and user behavior data is provided. The aggregation of the telemetry data and user presence events over time can provide sufficient context to understand user patterns and behavior as well as the user's environment. The provided system may learn from this data and recommend to the user best practices and actions to take to minimize the health risk specific to their behavior with respect to the computing ecosystem. In some instances, the provided system may initiate one or more actions, rather than a recommendation, that force the user to change their behavior if the user desires to continue interacting with the computing ecosystem.

### FIELD OF THE DISCLOSURE

The instant disclosure relates to information handling systems. More specifically, portions of this disclosure relate to an ergonomic behavioral aid for user interactions with a computing ecosystem.

### BACKGROUND

As the value and use of information continues to increase, individuals and businesses seek additional ways to process and store information. One option available to users is information handling systems. An information handling system generally processes, compiles, stores, and/or communicates information or data for business, personal, or other purposes thereby allowing users to take advantage of the value of the information. Because technology and information handling needs and requirements vary between different users or applications, information handling systems may also vary regarding what information is handled, how the information is handled, how much information is processed, stored, or communicated, and how quickly and efficiently the information may be processed, stored, or communicated. The variations in information handling systems allow for information handling systems to be general or configured for a specific user or specific use such as financial transaction processing, airline reservations, enterprise data storage, or global communications. In addition, information handling systems may include a variety of hardware and software components that may be configured to process, store, and communicate information and may include one or more computer systems, data storage systems, and networking systems.

One example application and associated data that may be executed by an information handling system is an application that generates an audio-visual experience for the user, such as a game application. Innovations and advancement in computer and video gaming technology for information handling systems enable gamers to play their favorite games anytime, anywhere and with any device. The availability of social and streaming platforms has provided the opportunity for gamers to build social communities, interact and earn a living from playing their favorite games.

### SUMMARY

The present application provides a data-driven, intelligent ergonomic behavioral aid based on uniquely captured telemetry and user behavior data. When a user is interacting with an application (e.g., playing a game) within their computing ecosystem (e.g., gaming ecosystem), data input and telemetry relating to user patterns and interactions are captured by the ecosystem. A presence service may capture user location events and changes from a controller and/or other wearable devices and perform location analysis to understand the user environment. An onboarding process may enable the system to understand the user's interaction range. The aggregation of the telemetry data and user presence events over time can provide sufficient context to understand user patterns and behavior as well as the user's environment. An information handling system according to embodiments of this disclosure may learn from this data and recommend to the user best practices and actions to take to minimize the health risk specific to their behavior with respect to the computing ecosystem. In some instances, the system may initiate one or more actions that force the user to change their behavior if the user desires to continue interacting with the computing ecosystem.

Computer or PC gaming may be a sedentary activity and may lead to numerous health problem—especially for regular gamers. Bad posture, eye strain, muscle and joint issues from repetitive hand movement, obesity, RSI and carpal tunnel are some health risks of regular gaming. Systems and techniques described herein may identify users gaming behavior based on user environment and habits and recommend best practices and actions to help them develop healthy gaming habits.

Aspects of embodiments of this disclosure allow a user to move between environments, move between devices, and/or move between services through automated configuration algorithms that may determine a location of the user and the device and migrate the device to an environment corresponding to the determined location. A knowledge graph may be used to support determinations regarding the devices, services, connections, and other aspects of an application session involving the user and a particular peripheral device.

The ergonomics of a user may be supported by an information handling system that uses available telemetry from multiple sources to build a confidence-based knowledge graph of the user's gaming environments and determine a position of the user within that graph as well as the user's posture and other ergonomic behaviors. A system with knowledge of devices in a user's gaming environment may build a knowledge graph by aggregating and comparing telemetry. For example, network telemetry may reveal that devices are positioned relatively near each other, a mobile device may reveal an absolute location based on GPS data, and/or an infrared presence sensor may reveal that the user is sitting in front a device. An intelligent system may assemble these individual pieces of telemetry into a broader knowledge graph based on the absolute and/or relative locations of the user's devices, the location of the user in relation, and or characteristics of the devices. This knowledge graph may be updated in real time and/or based on changes in device telemetry.

According to one embodiment, an information handling system includes at least one processor; and a memory coupled with the at least one processor and storing processor-readable code that, when executed by the at least one processor, is configured to perform operations including: receiving telemetry information associated with a user performing a first user session of an application; generating a health risk score of the user based on the telemetry information, the health risk score associated with a behavior of the user during the first user session; and initiating, in response to the health risk score meeting a first criteria, a first action associated with the first user session.

In certain embodiments, the first action includes pausing the first user session of the application for a predetermined amount of time.

In certain embodiments, the first action includes lowering a volume of sound emitted from a first device.

In certain embodiments, the first action includes transmitting a behavioral recommendation to a first device, the behavioral recommendation suggesting the user change the behavior.

In certain embodiments, the operations further include initiating, in response to the health risk score meeting a second criteria different than the first criteria, a second action associated with the first user session of the application, and the second action includes: terminating the first user session of the application at the first device; and initiating a second user session of the application at a second device.

In certain embodiments, the operations further include: retrieving a mapping of a plurality of devices to a plurality of locations; determining the second device for initiating the second user session based on the mapping of a plurality of devices to a plurality of locations and based on a first location for the first device being different from a second location for the second device.

In certain embodiments, determining the second device includes determining a mobile device as the second device at the second location for initiating the second user session based on the first device being a stationary device.

In certain embodiments, determining the second device is further based on the telemetry information, and wherein determining the second device includes determining, based on the telemetry information, that a head posture of the user associated with using the first device is different than a head posture of the user associated with using the second device.

In certain embodiments, determining the second device includes determining the second device based on information that the second device is at least one of positioned at a different height relative to the user than the first device or positioned further away from the user than the first device.

According to another embodiment, a method may include receiving telemetry information associated with a user performing a first user session of an application; generating a health risk score of the user based on the telemetry information, the health risk score associated with a behavior of the user during the first user session; and initiating, in response to the health risk score meeting a first criteria, a first action associated with the first user session.

In certain embodiments, the method may further include generating a behavior score of the user based on the received telemetry information, the behavior score associated with the behavior of the user during the first user session; and comparing the generated behavior score with a previously generated behavior score of the user, wherein the health risk score is generated in response to the behavior score being different than the previously generated behavior score.

In certain embodiments, the application is a video game system.

In certain embodiments, the method may further include detecting active devices associated with the first user session; retrieving a mapping of a plurality of devices to a plurality of locations, the plurality of devices including the active devices; and determining a position of the user relative to the active devices based on the retrieved mapping.

In certain embodiments, the method may further include retrieving a mapping of user furniture to a plurality of locations, and wherein generating the health risk score of the user is further based on the retrieved mapping of user furniture.

In certain embodiments, the first action comprises transmitting analytics to a first device, the analytics associated with the behavior of the user during the first user session.

According to another embodiment, a system may include a first device associated with a first user session of an application, the first device may generate telemetry information associated with a user performing the first user session; and a server that may: receive, from the first device, the telemetry information associated with the user performing the first user session of the application; generate a health risk score of the user based on the telemetry information, the health risk score associated with a behavior of the user during the first user session; and initiate, in response to the health risk score meeting a first criteria, a first action associated with the first user session.

In certain embodiments, the system may further include a second device, and the first action includes migrating the first user session of the application from the first device to the second device.

In certain embodiments, the server may further determine the second device based on the telemetry information received from the first device, and wherein determining the second device includes determining, based on the telemetry information, that a posture of the user associated with using the first device is different than a posture of the user associated with using the second device.

In certain embodiments, the server may further: retrieve a mapping of user furniture to a plurality of locations; determine a first seating apparatus associated with the first device based on the mapping; and determine the second device based on the telemetry information received from the first device and a second seating apparatus associated with the second device based on the mapping.

In certain embodiments, the application is a first application, and wherein the first action includes: terminating the first user session of the application on the first device; and initiating a second user session of a second application different than the first application on the first device

As used herein, the term “coupled” means connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially parallel includes parallel), as understood by a person of ordinary skill in the art.

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

For purposes of this disclosure, an information handling system may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, an information handling system may be a personal computer (e.g., desktop or laptop), tablet computer, mobile device (e.g., personal digital assistant (PDA) or smart phone), server (e.g., blade server or rack server), a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, touchscreen and/or a video display. The information handling system may also include one or more buses operable to transmit communications between the various hardware components.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus or system that “comprises,” “has,” or “includes” one or more elements possesses those one or more elements, but is not limited to possessing only those elements. Likewise, a method that “comprises,” “has,” or “includes,” one or more steps possesses those one or more steps, but is not limited to possessing only those one or more steps.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is a block diagram illustrating aspects of a configurable system for providing services to users according to some embodiments of the disclosure.

FIG. 2 is a block diagram illustrating possible game environments according to some embodiments of the disclosure.

FIG. 3A is a block diagram illustrating application and services hosted in different gaming environments according to some embodiments of the disclosure.

FIG. 3B is a block diagram illustrating application and services hosted in different gaming environments according to some embodiments of the disclosure.

FIG. 3C is a block diagram illustrating application and service hosted in a common gaming environment according to some embodiments of the disclosure.

FIG. 3D is a block diagram illustrating a cloud-based service arrangement for a gaming environment according to some embodiments of the disclosure.

FIG. 4 is a block diagram illustrating an example of a system for recommending or effectuating an ergonomic behavioral change in a user according to one or more aspects of the present disclosure.

FIG. 5 is a flow diagram of an example method for recommending or effectuating an ergonomic behavioral change in a user according to one or more aspects of the present disclosure.

FIG. 6 is a flow chart of an example method for initiating an action to help change a user's ergonomic behavior according to one or more aspects of the present disclosure.

FIG. 7 is a flow chart of an example method for effectuating an ergonomic behavioral change in a user according to one or more aspects of the present disclosure.

FIG. 8 is a block diagram illustrating the training and use of a knowledge graph of environments, users, and/or devices according to some embodiments of the disclosure.

FIG. 9 is a schematic block diagram of an example information handling system according to some embodiments of the disclosure.

### DETAILED DESCRIPTION

These example embodiments describe and illustrate various aspects of a data-driven, intelligent ergonomic behavioral aid, which may be an information handling system, that may recommend or effectuate an ergonomic behavioral change for a user interacting with a computing ecosystem based on uniquely captured telemetry and user behavior data. The behavioral change may place the user in a better ergonomic position to help reduce a risk to the user's health, such as reducing the likelihood of a repetitive strain injury. In one example, a user may interact with a gaming ecosystem such as for PC gaming or a gaming console. The telemetry information may be captured from a variety of sensors and other suitable data capturing methods included with the devices that make up the computing (e.g., gaming) ecosystem.

In some embodiments, a hub device may be located in a user's home and used to arrange game play sessions (or more generically application sessions) between host devices and services. The host devices may execute an application for receiving an AV stream for displaying rendered content from a game play session (or other application session), and in some configurations also receive user input for interacting with the session from a peripheral device, such as a gaming controller. The AV stream presented by the host device may be generated by a service. The service may execute on the hub device or another information handling system, such as a cloud computing resource. A home may include one or several host devices (e.g., televisions, mobile computers, tablet computers, and/or personal computers) and may include one or several information handling systems executing the service (e.g., a hub devices and/or personal computers).

The user's home may be divided into different environments defined by a space around a host device. For example, a living room with a television may be one environment and a bedroom with a personal computer may be another environment. A user may use a peripheral device in one of the environments and the hub device may configure a host device, a service, and the peripheral device for operation in the environment by determining the corresponding environment using a knowledge graph. The knowledge graph provides a database of historical information about the environments from which the hub device may use current characteristics of the peripheral device to deduce the location, and thus current environment, of the peripheral device. For example, the knowledge graph may include information about location of rooms (e.g., environments) in the house based on wireless signatures of devices within the different rooms. This difference in signatures reflects that a device on a one side of the house may receive beacon signals from different neighboring access points than a device on an opposite side of the house. When a user carries the peripheral device around the house, the hub device may determine a location of the peripheral device based on visible access points to the peripheral device. Other example characteristics beyond wireless signature for determining location are described in further detail below, and the knowledge graph may be used to combine different characteristics to identify the location, and thus environment, of the peripheral device.

In another example, the knowledge graph may include information about the user's setup in each of the rooms (e.g., environments). For instance, each room may have a place where the user sits (e.g., a chair, couch, etc.) and a screen that the user views (e.g., computer monitor, television, etc.). The distance between the seat and the screen may be different in different rooms. A height of the screen relative to the user in a respective seat may also be different between the rooms. Additionally, the user may maintain different postures depending on the seat in which the user is positioned. All of these example factors can influence the user's ergonomics and can therefore be used by the hub device for determining a risk to the user's health when the user is gaming in a particular room.

In some embodiments, based on the location of the peripheral device determined from the knowledge graph, the hub device may initialize an application session for the peripheral device by determining an appropriate host device and service for the application session. For example, if the peripheral device is in the living room and is requesting a game that is within the capabilities of the service on the hub device to execute, the hub device may initialize an application session for the peripheral device between the television as a consumption device and the hub device as a service. The service on the hub device executes the game and streams rendered content to an application executing on the television consumption device.

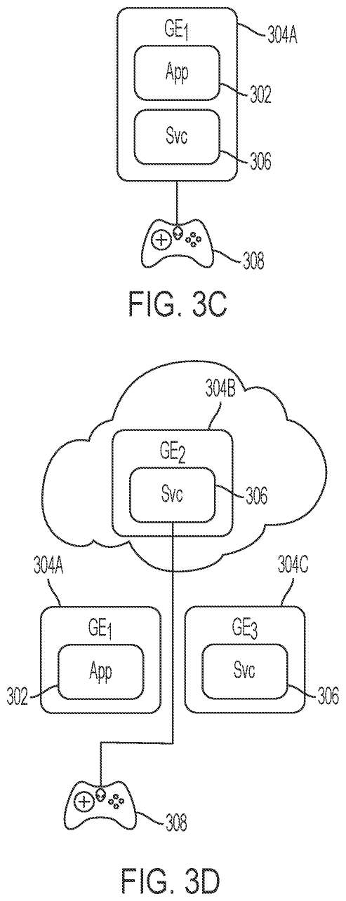

The hub device may be used to migrate the peripheral device to a different environment and/or migrate the application session between host devices and/or services. For example, initially the application session may use a communication link between the peripheral device and the television host device for receiving user input, in which the application executing on the television host device relays user input to the service through a backhaul communication link from the television host device to the hub device. During the application session, the hub device may monitor characteristics of the peripheral device, including signal strength of connection to other components, and determine that the communication link from the peripheral device to the hub device is stronger than the peripheral device to the television host device. The hub device may migrate the peripheral device to a communications link with the hub device such that the service executing on the hub device directly receives the user input but the streaming session continues from the service to the application executing on the television host device. Such a change is illustrated in the change in configuration from FIG. 3A to the configuration of FIG. 3B described in further detail below.

Other aspects of the application session may also be migrated. For example, if the peripheral device is determined to move to a different environment, then the hub device may migrate the application session to an application executing on a host device within the new environment. As another example, if a connection between the television host device and the service becomes unstable, the hub device may recommend and/or initiate a migration of the application session to a different host device. One scenario for such a migration may be where the television host device is connected through a wireless link to the service in which the wireless link quality is reducing quality of the streaming and a second host device with a wired connection is available in a nearby environment. Each of these example migrations may be determined based on information in the knowledge graph regarding locations of environments and capabilities within those environments. As yet another example, a user may request execution of an application, such as a particular game, during the application session for which a better configuration exists than the current host device and/or current service. The request for a different application, such as a game requiring a certain GPU capability, may cause the hub device to determine that a second device executing a second service is better for hosting the application and migrate the peripheral device to the second service by, for example, reconfiguring network connections.

In another example still, the hub device may determine that the user has been playing an application (e.g., game) in a particular environment for too long and therefore has an increased health risk. For instance, the user may be playing the game in the user's bedroom in which the screen is close to the user. In response, the hub device in some embodiments may transmit a message for display on the screen in the bedroom that recommends the user change environments to improve the user's ergonomics. In other embodiments, the hub device may terminate the application session in the bedroom and only allow the user to start a new application session, or continue the terminated application session, if the user moves to a new environment such as the living room in which the screen is farther away from the user. Upon the hub device determining that the user (e.g., the peripheral device) has moved to a different environment, the hub device may migrate the application session to an application executing on a host device within the new environment.

The hub device may support connecting to multiple peripheral devices. In one example, the hub device may support two peripheral devices using a shared session on one host device to play the same or different games on the host device. In another example, the hub device may support two peripheral devices in different environments using different sessions with different host devices. The hub device may determine the environment of each of the peripheral devices based on characteristics of the device and the knowledge graph and configure application session for each of the peripheral devices accordingly. Different arrangements of peripherals and players may be supported. For example, one hub device executing a service and one host device executing an application can support a configuration with Game A and one player (P1) with peripheral (C1) and Game B and one player (P2) with peripheral (C2); or can support a configuration with Game A and one player (P1) with peripheral (C1) and Game A and one player (P2) with peripheral (C2); or can support a configuration with Game A and two players (P1, P2) with peripherals (C1, C2).

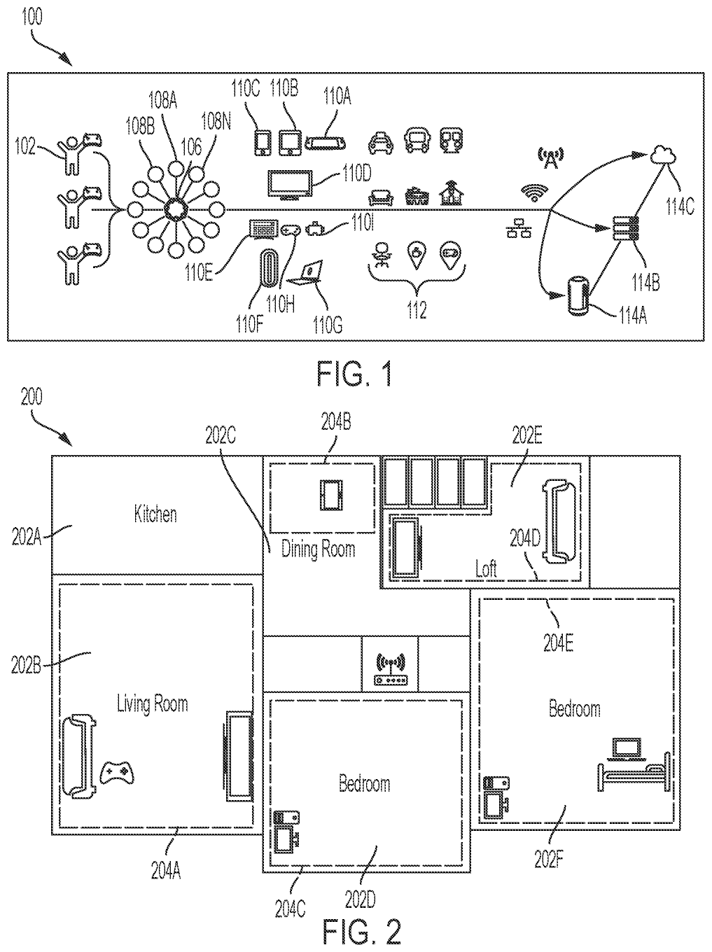

FIG. 1 is a block diagram illustrating aspects of a configurable system for providing services to users according some embodiments of the disclosure. A system 100 includes users 102 who may have access to a shared library of applications 106 including applications 108A-108N. The users 102 may have separate libraries, with some overlapping applications between the libraries. The users 102 may access the library 106 through devices 110A-I, such as mobile gaming device 110A, tablet computing device 110B, phone computing device 110C, television 110D, personal computing device 110E, desktop computing device 110F, laptop computing device 110G, game controller 110H, VR headset 110I. The devices 110 may access services at any of locations 112, including cars, busses, homes, hotels, offices, parks, etc. One or more of the devices 110 may communicate with an application session executing on a computing device 114, such as a home application hub 114A, a server 114B, or a cloud execution environment 114C. In some embodiments, environments may only exist for fixed devices, e.g., desktop computers, televisions, etc.

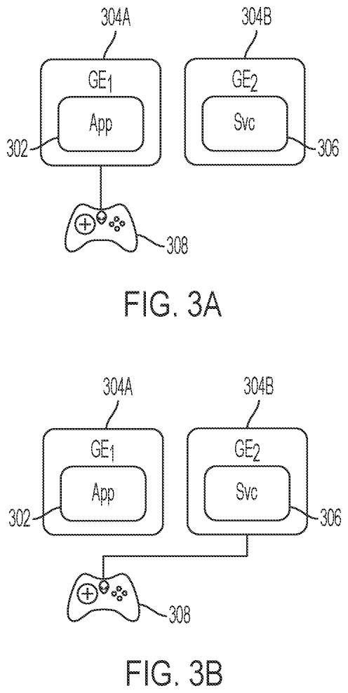

FIG. 2 is a block diagram illustrating possible game environments according to some embodiments of the disclosure. A user's home 200 may include rooms 202A-F, and each of the rooms may have different information handling systems present, different AV equipment present, and/or different characteristics. For example, a living room 202B may include a large-size television, a bedroom 202D may include a personal computer, and a dining room 202C may include a table computing device. Gaming environments 204A-E in the home 200 may be defined based on spaces where a user is likely to execute an application session. Each gaming environment 204A-E may include numerous devices and gaming environments, devices that may or may not be capable of hosting games, and/or devices that may or may not be capable of receiving game output. A system 100 may allow multiple users in the home 200 to simultaneously execute an application session. In some embodiments, multiple games may be hosted on a single device. In some embodiments, multiple games may target a single output device. In some embodiments, solution manages where games should be hosted, where game output should go, and how to best route peripheral I/O for users.

A user may move between gaming environments 204A-E within the home 200 and continue an application session. For example, a user may take a device, such as a gaming controller, from environment 204A to environment 204C. The gaming controller may migrate and reconfigure for operation in environment 204C from a configuration for environment 204A. For example, the controller may transition from an application hosted on a TV in living room 202B to an application hosted on TV in dining room 202C while remaining connected to a host service executing on a PC in bedroom 202D.

Example configurations for applications and services in gaming environments are shown in FIG. 3A-3D. FIG. 3A is a block diagram illustrating application and services hosted in different gaming environments according to some embodiments of the disclosure. In FIG. 3A, a first gaming environment 304A may include a device, such as a TV or PC, hosting an application 302, which is an endpoint for an application session such as a gaming session. The application 302 communicates with a service 306, which may be hosted on a device in a different gaming environment 304B. A controller 308 may communicate with the application 302 to receive user input for the application session to control, for example, a character in a game. In some embodiments, the controller 308 is connected to the environment 304A hosting the application and the I/O is configured to be relayed to the environment 304B hosting the actual game.

Another arrangement for the application and service is shown in FIG. 3B. FIG. 3B is a block diagram illustrating application and services hosted in different gaming environments according to some embodiments of the disclosure. In FIG. 3B, the controller 308 communicates with the service 306 for providing user input to an application session, with the AV rendering target of the application session being application 302 in a different gaming environment.

Another arrangement for the application and service is shown in FIG. 3C. FIG. 3C is a block diagram illustrating application and service hosted in a common gaming environment according to some embodiments of the disclosure. In FIG. 3C, the application 302 and the service 306 are executed in the same gaming environment 304A, which may be a single device, two devices, or a combination of devices in the gaming environment 304A. The controller 308 may communicate with either the service 306 and/or the application 302.

A further arrangement for the application and service is shown in FIG. 3D. FIG. 3D is a block diagram illustrating a cloud-based service arrangement for a gaming environment according to some embodiments of the disclosure. In FIG. 3D, the controller 308 may communicate with a service 306 hosted in a gaming environment 304B that is remote from the gaming environment 304A in which the application 302 is executing. The service 306 may be executing, for example, on a remote device, such as when the user's home includes the gaming environment 304B but the user is engaging with application 302 at a location on a different network from their home (e.g., at a friend's house). The service 306 may also or alternatively be executed, for example, on a cloud computing device available as a subscription service to the user.

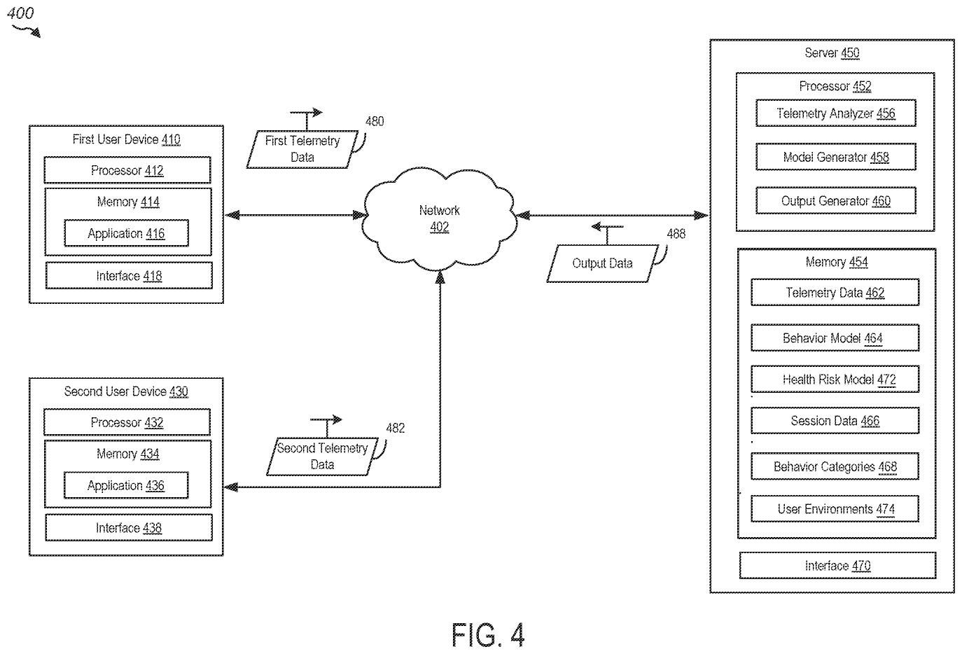

FIG. 4 is a block diagram illustrating a system 400 for recommending or effectuating an ergonomic behavior change for a user according to one or more aspects of the present disclosure. System 400 may include or correspond to system 100.

System 400 includes a network 402, a first user device 410, a second user device 430, and a server 450. Network 402 may include a wired network, a wireless network, or a combination thereof. To illustrate, network 402 may include a Bluetooth personal area network (PAN), a local area network (LAN), a wireless LAN (WLAN), a wide area network (WAN), a wireless network (e.g., a cellular network), a wired network, the Internet, one or more other networks, or a combination thereof, as illustrative, non-limiting examples.

First user device 410 and second user device 430 may include or correspond to one or more devices, such as device 110. To illustrate, first user device 410 or second user device 430 may include or correspond to a gaming console, a desktop computer, a laptop computer, a tablet, a mobile device, a peripheral device, a server (e.g., a cloud server), or another type of computing device, as illustrative examples. Additionally, or alternatively, first user device 410 or second user device 430 may include or correspond to an information handling system. In some implementations, first user device 410 or second user device 430 may be associated with an application environment, such as application environment 204, application environment 304, or a combination thereof.

First user device 410 may include one or more processors, such as a processor 412. Processor 412 may be a central processing unit (CPU) or other computing circuitry (e.g., a microcontroller, one or more application specific integrated circuits (ASICs), or the like) and may have one or more processing cores. First user device 410 may further include a memory 414 and an interface 418 (e.g., a wireless interface or a wired interface). Processor 412 may be coupled to memory 414, interface 418, or a combination thereof.

Memory 414 may include a read only memory (ROM) device, a random access memory (RAM) device, one or more hard disk drives (HDDs), a flash memory device, a solid state drives (SSDs), another devices configured to store data in a persistent or non-persistent state, or a combination of different memory devices. Memory 414 includes one or more instructions, such as an application 416. Application 416 may include or correspond to application 302. To illustrate, memory 414 may store instructions that, when executed by processor 412, cause processor 412 to perform operations as described herein. Additionally, memory 414 may store one or more thresholds, data, preferences, other settings, a combination thereof, or the like, as illustrative, non-limiting examples.

Interface 418 includes one or more communication interfaces. For example, interface 418 may include a long range (LoRa) interface, a Wi-Fi interface (e.g., an Institute of Electrical and Electronics Engineers (IEEE) 802.11 interface), a cellular interface (e.g., a fourth generation (4G) or long term evolution (LTE) interface, a fifth generation (5G) new radio (NR) interface, or the like), a Bluetooth interface, a Bluetooth low energy (BLE) interface, a Zigbee interface, a non-LoRa interface, another type of network interface, a combination thereof, or the like.

In some implementations, first user device 410 may include one or more additional devices or components. For example, first user device 410 may include an I/O device, such as one or more switches (e.g., depressible buttons, triggers, or the like), directional pads, one or more touchscreens, a microphone, a camera, one or more speakers, one or more light sources, vibration devices, other types of devices, or a combination thereof, as illustrative, non-limiting examples.

Second user device 430 may include or correspond to first user device 410. Second user device 430 may include one or more processors, such as a processor 432. Processor 432 may include or correspond to processor 412. Processor 432 may be a CPU or other computing circuitry (e.g., a microcontroller, one or more ASICs, or the like) and may have one or more processing cores. Second user device 430 may further include a memory 434 and an interface 438 (e.g., a wireless interface or a wired interface). Processor 432 may be coupled to memory 434, interface 438, or a combination thereof. Memory 434 and interface 438 may include or correspond to memory 414 and interface 418, respectively.

Memory 434 may include a ROM device, a RAM device, one or more HDDs, a flash memory device, an SSD, or other devices configured to store data in a persistent or non-persistent state, or a combination of different memory devices. Memory 434 includes one or more instructions, such as an application 436. Application may include or correspond to application 416. Memory 434 may store instructions that, when executed by processor 432, cause processor 432 to perform operations as described herein. Additionally, memory 434 may store one or more thresholds, data, preferences, other settings, a combination thereof, or the like, as illustrative, non-limiting examples.

Interface 438 includes one or more communication interfaces. For example, interface 438 may include a LoRa interface, a Wi-Fi interface (e.g., an IEEE 802.11 interface), a cellular interface (e.g., a 4G or LTE interface, a 5G NR interface, or the like), a Bluetooth interface, a BLE interface, a Zigbee interface, a non-LoRa interface, another type of network interface, a combination thereof, or the like.

In some implementations, second user device 430 may include one or more additional devices or components. For example, second user device 430 may include an I/O device, such as one or more switches (e.g., depressible buttons, triggers, or the like), directional pads, one or more touchscreens, a microphone, a camera, one or more speakers, one or more light sources, vibration devices, other types of devices, or a combination thereof, as illustrative, non-limiting examples.

In some implementations, a user device (e.g., 410 or 430) includes or is coupled to one or more peripheral devices. The one or more peripheral device may include or correspond to a game controller (e.g., a gamepad or a joystick controller), a mouse, a keyboard, a display, a speaker, a microphone, a headset, a virtual reality (VR) device, an augmented reality (AR) device, a vehicle control simulation device (such as a racing wheel or a vehicle pedal assembly), a musical instrument simulation device (such as a guitar controller), a detachable controller, or a brain-computer interface (BCI) device, as illustrative examples. In some implementations, a peripheral device of the one or more peripheral devices may correspond to a streaming device configured to stream content, such as gaming content or metaverse content, from the user device (e.g., 410 or 430). For example, the peripheral device may correspond to a handheld gaming device, a phone, a tablet, or a computer that streams gaming content from the user device (e.g., 410 or 430).

A peripheral device of the one or more peripheral device may include a processor, a memory, an interface, or a combination thereof. Additionally, or alternatively, the peripheral device may include one or both of an input device that receives user input (such as user operation of a button or other input device) or an output device that generates an output (such as one or more of an audio output, a visual output, a haptic output, or another output). To illustrate, the peripheral device may be configured to receive user input and to transmit signals to the user device (e.g., 410 or 430) based on the user input (e.g., via a wired or wireless connection to the user device). Additionally, or alternatively, the peripheral device may be configured to receive commands from the user device (e.g., 410 or 430) (e.g., via a wired or wireless connection to the user device) and to generate an output (such as one or more of an audio output, a visual output, a haptic output, or another output) based on the commands.

Server 450 may include or correspond to computing device 114. In some implementations, server 450 is configured to host an application session or provide a service. Additionally, or alternatively, server 450 may include or correspond to an information handling system. In some implementations, server 450 is configured as a cloud server or a cloud execution environment.

Server 450 may include one or more processors, such as a processor 452. Processor 452 may be a CPU or other computing circuitry (e.g., a microcontroller, one or more ASICs, or the like) and may have one or more processing cores. Server 450 may further include a memory 454 and an interface 470 (e.g., a wireless interface or a wired interface). Processor 452 may be coupled to memory 454, interface 470, or a combination thereof.

In some implementations, processor 452 may include modules having hardware, software, or a combination of hardware and software and be configured as a telemetry analyzer 456, a model generator 458, an output generator 460, or a combination thereof. Although described as being included in processor 452, in other implementations, telemetry analyzer 456, model generator 458, output generator 460, or a combination thereof, may additionally or alternatively be modules included in and/or modules executed by processor 412 or processor 432.

Telemetry analyzer 456 may receive telemetry data from individual systems during or after execution of applications, including game applications. In some embodiments, the telemetry data may be aggregated and correlated in a cloud system. In some embodiments, the telemetry data may be aggregated and correlated in a local system, such as the gaming hub in a user's home environment. The telemetry analyzer 456 may analyze the telemetry data to parse the telemetry information into separate sets of data and provide the parsed telemetry data and/or other representations of the telemetry information to the model generator 458. For example, telemetry information may indicate a user's posture during an application session. As another example, the telemetry information may indicate an action performed by the user (e.g., number of mouse clicks). A further example for analyzing telemetry data may include determining how focused a user is during an application session such as by analyzing, for example, window focus, mouse position, or amount of inactive time. A further example still is the telemetry information may indicate how far the user is away from a screen. Regardless of what is determined from the telemetry information, the information may be provided to model generator 458.

These examples may provide some useful information regarding user ergonomics from the telemetry data. For example, from the information indicating a user's posture it may be determined whether the user has held a static posture for too long and is at risk of developing muscle imbalances that contribute to bad posture. In another example, from the information indicating an action performed by the user it may be determined what the user's risk is for developing a repetitive strain injury. In another example still, from the information indicating how focused a user is it may be determined that the user has been stationary for too long which can contribute to weight gain. In another example, from the information indicating how far the user is away from a screen it may be determined what the user's risk is for straining the user's eyes.

The telemetry analyzer 456 may generate behavior scores indicative of a user's behaviors and health risk scores indicative of a risk the user has of developing a health issue based on the telemetry data, or a model generator 458 may be trained with the telemetry data to generate one or more models for generating the behavior scores and health risk scores from the telemetry data. For example, a model may be updated by adjusting the weights assigned to different correlated events. When the model generator 458 executes on a user's home application hub, the model may be trained to generate behavior scores and health risk scores based on a particular user's behavior. For example, the telemetry data and user presence events accumulating over time will provide sufficient context to understand user gaming patterns and behavior as well as the user's gaming environment. The model generator 458 may be used in combination with the telemetry analyzer 456 to generate behavior scores and health risk scores.

Behavior scores and health risk scores and/or other information output from telemetry analyzer 456 and model generator 458 may be provided to output generator 460. The output generator 460 may produce output, such as through a graphical user interface (GUI) to a user, to indicate information about the user's ergonomics during an application session. For example, the output generator 460 may display a reward (e.g., a “keep up the good work” graphical message) in response to the user's behaviors not increasing the user's risk for health issues. In another example, the output generator 460 may display certain behavior metrics that indicate to a user that their behavior is trending in the wrong direction. As another example, the output generator 460 may display a recommendation that the user change one or more behaviors since it has been determined their risk for developing a health issue has increased.

In some embodiments, the output of the output generator 460 initiates an action in order to effectuate a change in the user's behavior. For example, the output generator 460 may terminate an application session in the user's current environment and require the user to move to a different environment if the user desires to continue the application session. In another example, the output generator 460 may reduce the volume of sound emitted from speakers. In another example still, the output generator 460 may remap a game controller so the user has to use a different hand or a different finger(s). Further examples of output from output generator 460 may include pausing an application session for a predetermined or custom amount of time, switching the application, and changing the screen on which the application is displayed within the same environment.

Memory 454 may include a ROM device, a RAM device, one or more hard disk drives (HDDs), a flash memory device, an SSD, another device configured to store data in a persistent or non-persistent state, or a combination of different memory devices. Memory 454 includes one or more instructions, such as an application or a service. The application may include or correspond to application 416 or 436. The service may include or correspond to service 306. To illustrate, memory 454 may store instructions that, when executed by processor 452, cause processor 452 to perform operations as described herein.

Additionally, memory 454 may store one or more thresholds, data, preferences, other settings, a combination thereof, or the like, as illustrative, non-limiting examples. For example, memory 454 may include or be configured to store telemetry data 462, a behavior model 464, a health risk model 472, session data 466, behavior categories 468, user environments 474, or a combination thereof. Although memory 454, and server 450, are described as including each of telemetry data 462, a behavior model 464, a health risk model 472, session data 466, behavior categories 468, user environments 474, in other implementations, telemetry data 462, a behavior model 464, a health risk model 472, session data 466, behavior categories 468, user environments 474, or a combination thereof, may be stored at a memory or database that is separate from and accessible to server 450. The behavior model 464 and/or the health risk model 472 may be machine learning models.

Interface 470 includes one or more communication interfaces. For example, interface 470 may include a LoRa interface, a Wi-Fi interface (e.g., an IEEE 802.11 interface), a cellular interface (e.g., a 4G or LTE interface, a 5G NR interface, or the like), a Bluetooth interface, a BLE interface, a Zigbee interface, a non-LoRa interface, another type of network interface, a combination thereof, or the like.

During operation, system 400 may involve data transfer between the server 450, which may be the user application hub (such as a home gaming hub) or a cloud-based server, and one or more user devices 410 and 430. Operations performed by the system 400 may include training a behavior score model and/or a health risk model in model generator 458 based on telemetry information processed by telemetry analyzer 456 including ecosystem telemetry (e.g., application sessions, user information) and system telemetry (e.g., CPU utilization, GPU utilization, memory loads). The processor 452 may determine, based on the behavior model 464, a behavior score for a particular user behavior indicative of how ergonomically sound the behavior is, and whether that behavior score is different from a previously determined behavior score. In response to the behavior score being different, the processor 452 may determine a risk of developing health issues for the user based on the health risk model 472. The processor 452 may then determine information or a recommendation, or an action to initiate, or other output from the output generator 460, for the user based on the health risk model 472.

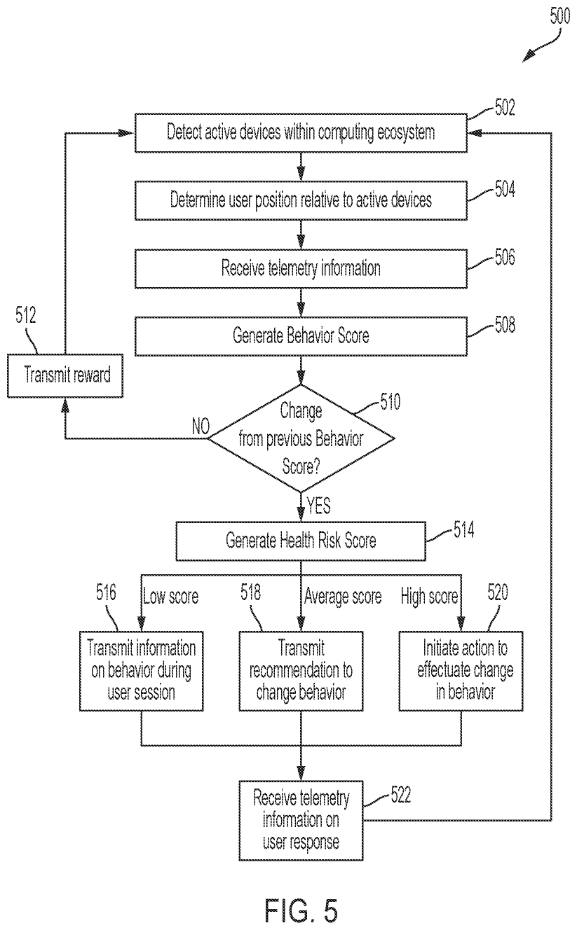

FIG. 5 illustrates a flow diagram of an example method 500 for recommending or effectuating an ergonomic behavioral change in a user. In some aspects, the method 500 includes at block 502 detecting active devices within a computing (e.g., gaming) ecosystem. For example, a user may start an application (e.g., game) executing on their computer in their bedroom and connect to a service executing on a hub device, and the hub device may detect the devices (e.g., monitor, mouse, keyboard, speakers, wearable device, etc.) that the user is actively using with the game. For instance, in some embodiments, the hub device may detect the devices from which the hub device has received a connection request.

Embodiments of this disclosure provide for determining a configuration of an environment around a user based on, for example, determining a location of the user with respect to devices and environments. At block 504, a position of a user may be determined relative to the active devices. For example, characteristics of each device may be determined. Characteristics may include one or more of physical connections, physical location (e.g., GPS coordinates), IP address, Bluetooth devices in range, ultra-wideband connections available, ultrasonics detected, access points (APs) in range, infrared sensor received data, camera images, microphone audio input, biometric sensor input, eye tracking input, accelerometer input, gyroscope input, device metadata (e.g., type of device, brand of device, model number of device, primary user of the device, capabilities of the device, etc.), and user behavior.

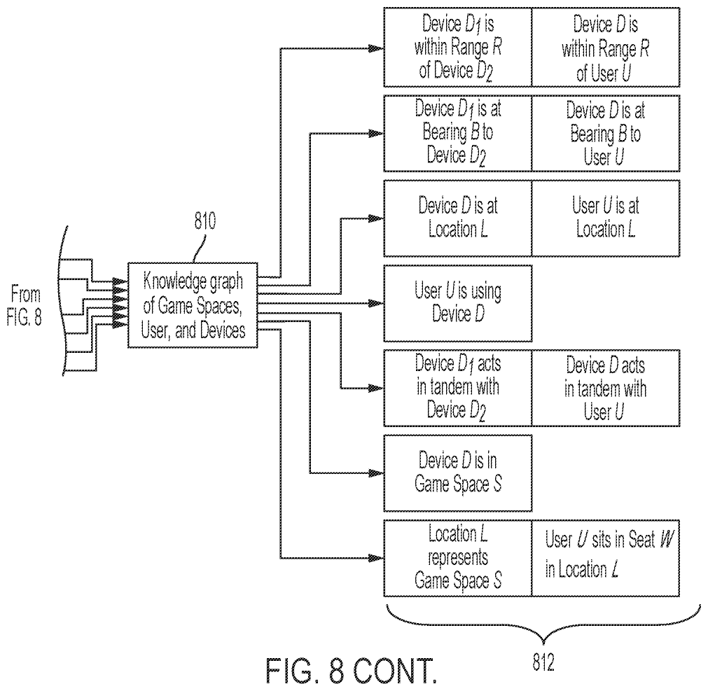

An environment of the active devices may be determined based on the determined device characteristics and a knowledge graph. The environment may be a physical location or a relative location to other devices and/or the user. The determining of the environment may include determining a configuration of devices for executing an application and a service. The knowledge graph may be a database of environments, users, and devices. The knowledge graph may be a static database created by a user. The knowledge graph may alternatively be a dynamic database that is trained over time using inputs and feedback from devices. The knowledge graph may be stored on a hub device (e.g., the server 450), such as a hub in the user's home and/or a hub hosted in the cloud for the user. The knowledge graph may be constructed as described below in aspects of FIG. 8. The determination of the environment of the active devices may include, for example, one or more of determining a device D1 is within range R of another device D2, determining device D is within range R of user U, determining user U is within range R of user U2, determining device D1 is at bearing B to device D2, determining device D is at bearing B to user U, determining user U1 is at bearing B to user U2, determining device D is at location L, determining user U is at location L, determining user U is using device D, determining device D1 acts in tandem with device D2, determining device D acts in tandem with user U, determining device D is in space S, and/or determining location L represents space S.

Based on the determined characteristics of each device and the stored information on known user environments in the knowledge graph, characteristics of the user's ergonomics can be inferred. For example, it can be determined that the user is in their bedroom at their desk in front of their computer based on the locations of the active devices relative to one another. It can then be inferred that the user is a certain distance from the screen, is using a mouse, is sitting in a certain chair, etc. In some embodiments, information from a wearable device of the user may help indicate an inferred posture of the user.

At block 506, the method 500 includes receiving telemetry information. For instance, the hub device may receive telemetry information from one or more of the active devices. In various examples, the telemetry information may include gaming ecosystem telemetry information, in-game user behavior, direct feedback from the user, motion input, biometric data, user preferences, etc. Gaming ecosystem telemetry information may include a user's overall time spent gaming, time spent on a particular game, amount of time the game is paused, information on a game (e.g., type of game), a response by the user to a previous recommendation to change their behavior, etc. In-game user behavior may include user focus (e.g., window focus, mouse position, inactivity), user posture (e.g., gaze, eye tracking, head position, body posture), user input (e.g., controller input, keyboard input, mouse input), etc. Direct feedback from the user may include survey responses, responses to questions in an onboarding process, etc. Motion input may include user location, the user's distance from a screen, altitude of user's location, etc. Biometric data may include mirror metrics (e.g., from a camera), fitness and health information (e.g., from wearable devices), etc. User preferences may include game settings, device settings, time spent playing on each device, etc.

At block 508, a behavior score may be generated based on the received telemetry information. For example, each received telemetry input may be mapped to a behavior category (e.g., distance from screen, head posture, body posture, hand movement, time spent playing, etc.). Each behavior category may be a feature of a model (e.g., the behavior model 464). The behavior score generated using the behavior model 464 may rank the user's degree of conformity to each of the behavior categories. In at least some embodiments, the behavior model 464 is a ranking algorithm that uses input from prior expert knowledge of mapped behaviors to pre-defined health risk. In some embodiments, a behavior score for each behavior category may be generated. In other embodiments, a behavior score is generated that encompasses every behavior category.

At diamond 510, the generated behavior score may be compared to a previously generated behavior score. At block 512, if the generated behavior score is the same as the previously generated behavior score, a reward may be transmitted. For example, the hub device may transmit a congratulatory message for display to a device in the gaming ecosystem to help reinforce to the user that they are doing well with their ergonomics. Other suitable rewards may include a monetary reward, points for a point-based system, unlocking an item in the game, etc. In some embodiments, a generated behavior score is deemed the same as the previously generated behavior score if the generated behavior score is within a predetermined deviation from the previously generated behavior score. If a reward is transmitted at block 512, the method 500 may return to block 502 in order to continue monitoring the user's behavior.

At block 514, if the generated behavior score is different than the previously generated behavior score, a health risk score may be generated (e.g., using the health risk model 472). The health risk score generated using the health risk model 472 is calculated based on how a user behavior relates to a potential health risk (e.g., bad posture, eye strain, muscle and joint issues from repetitive hand movement, obesity, RSI and carpal tunnel, etc.). In at least some embodiments, the health risk model 472 is a classifier algorithm that uses captured behavior (e.g., the telemetry information) and the generated behavior score to predict (e.g., confidence) a potential health risk in the form of the health risk score.

Based on the health risk score generated, the method 500 may proceed in different ways. At block 516, if the generated health risk score fails to meet a first predetermined threshold (e.g., a first criteria indicating a low score), then information on the user's behavior during the user session may be transmitted. For example, the hub device may transmit user behavior insights/analytics for display to a device in the gaming ecosystem. The user behavior insights/analytics may include a potential risk ratio for the user, user game behavior analytics, a distance the user covered while playing over a period of time, behavioral thresholds for an associated health risk compared to the user's level, etc.

At block 518, if the generated health risk score meets the first predetermined threshold but fails to meet a second predetermined threshold (e.g., a second criteria indicating an average score), then a recommendation to change the behavior may be transmitted. For example, the hub device may transmit the recommendation for display to a device in the gaming ecosystem. The recommendation may be any suitable change in behavior for the user to improve their ergonomics and reduce their likelihood for developing a health issue. For example, a recommendation may be to move further from the screen in a game space or to a different game space to ease eye strain, to change body posture based on what is available (e.g., move from the couch to a chair, lay down on the couch, stand up, move to a different room with a different seating arrangement), to remap repeatedly used buttons to mitigate repetitive motion injury, to map keys to optimize finger travel, to change games to reduce aggressive behavior and/or emotional strain, to change to a mobile device to encourage mobility.

In one particular example, a user has been playing a game for three hours in the user's bedroom during an application session. It is known from the knowledge graph that the user typically sits on a seat without a backrest when playing in the user's bedroom. From the knowledge graph and received telemetry information, a hub can infer the user's posture over the application session. The hub may generate a health risk score that indicates the user is at risk of developing a back ache due to bad posture. In response, the hub may transmit a recommendation for display to the user that suggests the user switch to gaming in the living room so that the user may sit on the couch.

At block 520, if the generated health risk score meets the second predetermined threshold (e.g., a third criteria indicating a high score), which is greater than the first predetermined threshold, then an action may be initiated to effectuate a change in the user's behavior. In one example, a user has been playing a game in the user's bedroom for two hours. It is known from the knowledge graph that the user typically sits a certain distance away from the screen when playing in the user's bedroom. From the knowledge graph and received telemetry information, the hub device determines that the user's risk for eye strain (e.g., health risk score) meets the second predetermined threshold requiring an action to be initiated. Using the knowledge graph, the hub device may determine that the user's living room has the greatest distance between a screen and the user. As such, the hub device may terminate or pause the user's game session in the bedroom and request that the user continue playing in the living room. The user can then proceed to the living room and the hub device can migrate the session to the living room so that the user can continue playing. In this way, the hub device effectuates a change in the user's behavior by requiring the user to move to a different location if the user desires to continue playing.

In another example, the hub device may pause the game the user is playing for a predetermined amount of time (e.g., five minutes) to give the user time to walk around. In another example, the hub device may terminate the game session the user is playing and start a session of a different game to allow the user to relax. In another example still, the hub device may terminate or pause the game session the user is playing and request that the user continue playing on a mobile device. In another example, the hub device may terminate or pause the game session the user is playing and request that the user continue playing in a different room that has a screen at a different height relative to the user in order to change the user's posture while playing. For instance, a screen at a different height can cause the user's head posture to change (e.g., looking straight ahead at the screen with a neutral cervical spine rather than looking upward or downward at a screen with cervical spine flexion or extension).

At block 522, telemetry information capturing the user's response to the transmitted information, transmitted recommendation, or initiated action may be received. For example, the hub device may receive telemetry information indicating that the user switched to gaming in the living room after a recommendation to do so was displayed to the user. The telemetry information capturing the user's response can be an input for future learning and can also serve as game developer feedback. The method 500 may then return to block 502 in order to continue monitoring the user's behavior.

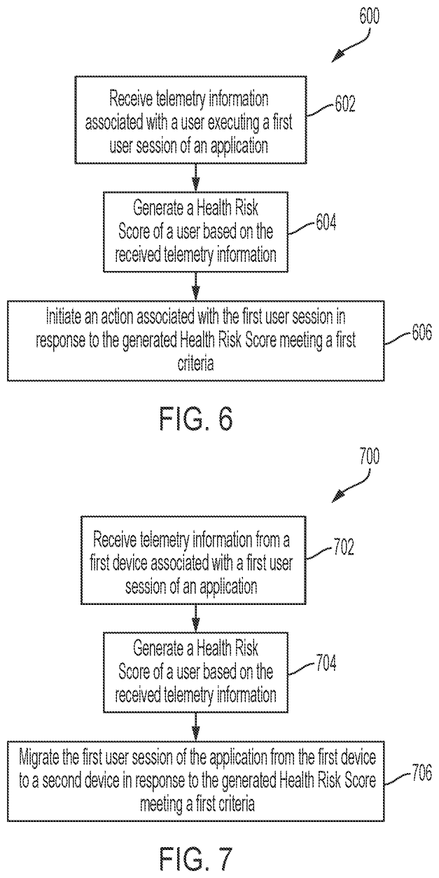

FIG. 6 illustrates a flow chart of an example method 600 for initiating an action to help change a user's ergonomic behavior. At block 602, the method 600 may include receiving telemetry information associated with a user performing a first user session of an application. For example, a hub device may receive telemetry information from one or more input controllers or periphery devices of a gaming ecosystem. At block 604, a health risk score of the user may be generated based on the received telemetry information. For example, the health risk score may be calculated using the health risk model 472. The health risk score generated using the health risk model 472 is calculated based on how a user behavior relates to a potential health risk (e.g., bad posture, eye strain, muscle and joint issues from repetitive hand movement, obesity, RSI and carpal tunnel, etc.). At block 606, an action associated with the first user session may be initiated in response to the generated health risk score meeting a first criteria. For example, the hub device may transmit a recommendation to pause the game and walk around for ten minutes, and the recommendation may be displayed on the screen the user is gaming with. In another example, the hub device may pause the game for ten minutes and not allow the user to continue playing until the time has elapsed to enforce a break for the user. In another example, the hub device may lower the volume of the speakers with which the user is gaming in response to determining that the user is at risk of damaging the user's hearing.

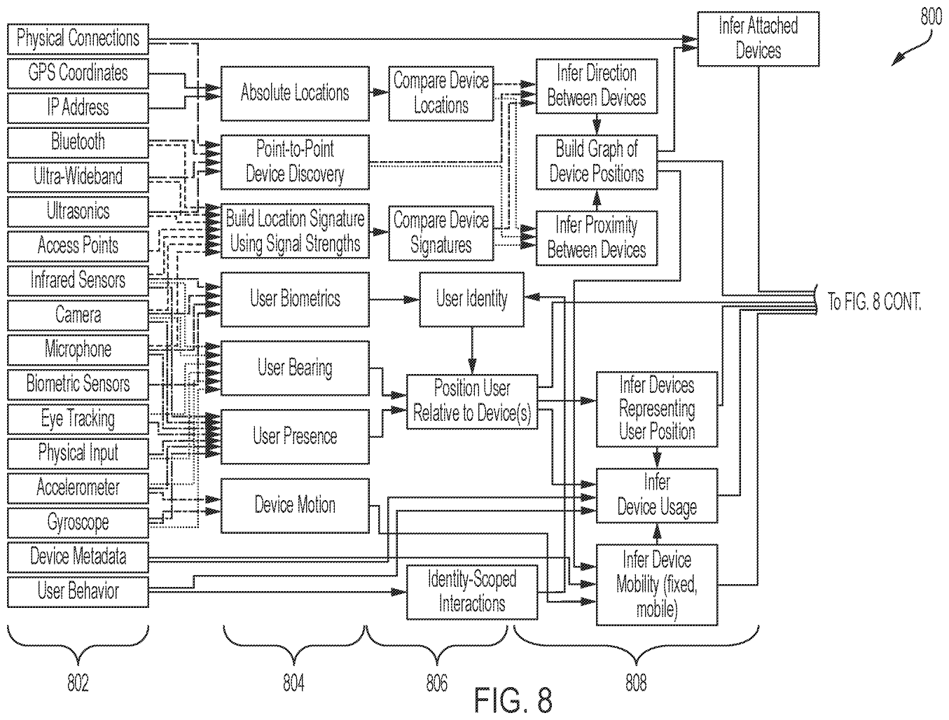

FIG. 7 illustrates a flow chart of an example method 700 for effectuating an ergonomic behavioral change in a user. At block 702, the method 700 may include receiving telemetry information from a first device associated with a first user session of a first application, the telemetry information associated with a user. For example, a hub device may receive telemetry information from one or more input controllers or periphery devices. At block 704, a health risk score of the user may be generated based on the received telemetry information. For example, the health risk score may be calculated using the health risk model 472. The health risk score generated using the health risk model 472 is calculated based on how a user behavior relates to a potential health risk (e.g., bad posture, eye strain, muscle and joint issues from repetitive hand movement, obesity, RSI and carpal tunnel, etc.). For example, the health risk score may be a normalized value in the range of 1-100 that provides a relative measure of user health against a cohort, in which the cohort may be users of a particular application, users with a similar device configuration, and/or users with similar demographic information (e.g., age, height, gender, weight, etc.). At block 706, the first user session of the application is migrated from the first device to a second device in response to the generated health risk score meeting a first criteria. For example, the hub device may migrate a gaming session from a computer monitor in the user's bedroom to a TV in the user's living room. As a result, the user must move to the living room to continue playing the game, which effectuates a change in the user's ergonomics since the user's setup is different in the living room as compared to the bedroom.

As noted in connection with FIG. 5 above, FIG. 8 is a block diagram illustrating the training and use of a knowledge graph of game environments, users, and/or devices according to some embodiments of the disclosure. A system 800 may include input data 802 processed through blocks 804 and blocks 806 and blocks 808 to train a knowledge graph 810 or another representation as a mapping of a plurality of devices to a plurality of locations. The input data 802 and resulting processed data in blocks 804, 806, and 808 are examples of telemetry data that may be used to determine actions to initiate in order to help improve a user's ergonomics. The knowledge graph 810 may be trained to identify relationships between gaming environments, users, and/or device as shown in outcomes 812. Although many example inputs, processes, and outcomes are shown in FIG. 8, a knowledge graph 810 may be trained with different combinations of the illustrated examples.

Examples of input data 802 regarding a device may include one or more of physical connections, physical location (e.g., GPS coordinates), IP address, Bluetooth devices in range, ultra-wideband connections available, ultrasonics detected, access points (APs) in range, infrared sensor received data, camera images, microphone audio input, biometric sensor input, eye tracking input, accelerometer input, gyroscope input, device metadata (e.g., type of device, brand of device, model number of device, primary user of the device, capabilities of the device, etc.), and user behavior.

The input data 802 collected by or from a device may be processed in blocks 804 to determine one or more of an absolute location, point-to-point discovery, a location signature using signal strengths of nearby devices, user biometrics, user bearing, user presence, and/or device motion. The processing at blocks 804 may be inputs to further processing at blocks 806 that includes, for example, comparing device locations, comparing device signatures, determining a user identity, and/or determining a position of user relative to device(s). The output of processing at blocks 804 and/or 806 may be input to further processing of blocks 808. The processing at blocks 808 may include, for example, determining a direction between devices, determining a graph of device positions, determining proximity between devices, determining devices representing user position, determining device usage, and/or determining mobility (e.g., whether a device is fixed or mobile). The output of processing blocks 808 may be used to train a knowledge graph 810. The knowledge graph 810 may be determined, for example, for users subscribed to a common hub, users in a common area, or an otherwise specified list of users. The knowledge graph 810 may be a database associating items, such as devices with users, users with other users, users with gaming environments, devices with gaming environments, users with devices, etc. In some embodiments, the knowledge graph 810 may be neural network or other artificial learning system trained using the input from blocks 802,804, 806, and 808 and/or with feedback from users.

The knowledge graph 810 may be used to determine outcomes 812. For example, outcomes 812 may include determining a device D1 is within range R of another device D2, determining device D is within range R of user U, determining device D1 is at bearing B to device D2, determining device D is at bearing B to user U, determining device D is at location L, determining user U is at location L, determining user U is using device D, determining device D1 acts in tandem with device D2, determining device D acts in tandem with user U, determining device D is in game space S, determining location L represents game space S, and/or determining user U is seated in seat W in location L. The outcomes 812 may be used to determine a user's environment (e.g., living room, bedroom, etc.) including the user's positioning relative to other devices in the environment. The user's ergonomics may be inferred based on the user's determined environment and the known furniture, devices, etc. in the determined environment.

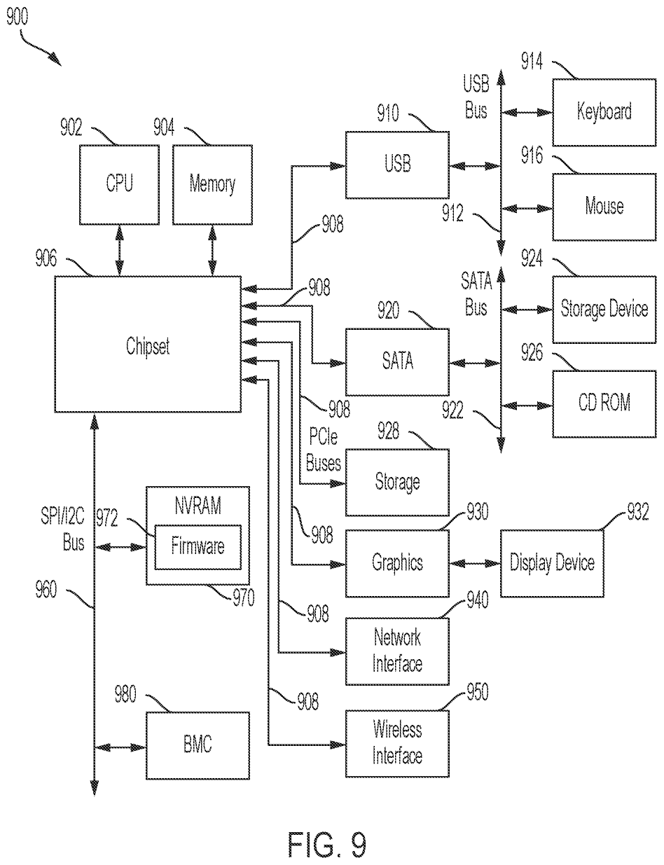

FIG. 9 illustrates an example information handling system 900, such as for a home gaming hub as described in embodiments above and other apparatus for executing the methods described above. Information handling system 900 may include a processor 902 (e.g., a central processing unit (CPU)), a memory (e.g., a dynamic random-access memory (DRAM)) 904, and a chipset 906. In some embodiments, one or more of the processor 902, the memory 904, and the chipset 906 may be included on a motherboard (also referred to as a mainboard), which is a printed circuit board (PCB) with embedded conductors organized as transmission lines between the processor 902, the memory 904, the chipset 906, and/or other components of the information handling system. The components may be coupled to the motherboard through packaging connections such as a pin grid array (PGA), ball grid array (BGA), land grid array (LGA), surface-mount technology, and/or through-hole technology. In some embodiments, one or more of the processor 902, the memory 904, the chipset 906, and/or other components may be organized as a System on Chip (SoC).

The processor 902 may execute program code by accessing instructions loaded into memory 904 from a storage device, executing the instructions to operate on data also loaded into memory 904 from a storage device, and generate output data that is stored back into memory 904 or sent to another component. The processor 902 may include processing cores capable of implementing any of a variety of instruction set architectures (ISAs), such as the x86, POWERPC®, ARM®, SPARC®, or MIPS® ISAs, or any other suitable ISA. In multi-processor systems, each of the processors 902 may commonly, but not necessarily, implement the same ISA. In some embodiments, multiple processors may each have different configurations such as when multiple processors are present in a big-little hybrid configuration with some high-performance processing cores and some high-efficiency processing cores. The chipset 906 may facilitate the transfer of data between the processor 902, the memory 904, and other components. In some embodiments, chipset 906 may include two or more integrated circuits (ICs), such as a northbridge controller coupled to the processor 902, the memory 904, and a southbridge controller, with the southbridge controller coupled to the other components such as USB 910, SATA 920, and PCIe buses 908. The chipset 906 may couple to other components through one or more PCIe buses 908.

Some components may be coupled to one bus line of the PCIe buses 908, whereas some components may be coupled to more than one bus line of the PCIe buses 908. One example component is a universal serial bus (USB) controller 910, which interfaces the chipset 906 to a USB bus 912. A USB bus 912 may couple input/output components such as a keyboard 914 and a mouse 916, but also other components such as USB flash drives, or another information handling system. Another example component is a SATA bus controller 920, which couples the chipset 906 to a SATA bus 922. The SATA bus 922 may facilitate efficient transfer of data between the chipset 906 and components coupled to the chipset 906 and a storage device 924 (e.g., a hard disk drive (HDD) or solid-state disk drive (SDD)) and/or a compact disc read-only memory (CD-ROM) 926. The PCIe bus 908 may also couple the chipset 906 directly to a storage device 928 (e.g., a solid-state disk drive (SDD)). A further example of an example component is a graphics device 930 (e.g., a graphics processing unit (GPU)) for generating output to a display device 932, a network interface controller (NIC) 940, and/or a wireless interface 950 (e.g., a wireless local area network (WLAN) or wireless wide area network (WWAN) device) such as a Wi-Fi® network interface, a Bluetooth® network interface, a GSM® network interface, a 3G network interface, a 4G LTE® network interface, and/or a 5G NR network interface (including sub-6 GHz and/or mmWave interfaces).

The chipset 906 may also be coupled to a serial peripheral interface (SPI) and/or Inter-Integrated Circuit (I2C) bus 960, which couples the chipset 906 to system management components. For example, a non-volatile random-access memory (NVRAM) 970 for storing firmware 972 may be coupled to the bus 960. As another example, a controller, such as a baseboard management controller (BMC) 980, may be coupled to the chipset 906 through the bus 960. BMC 980 may be referred to as a service processor or embedded controller (EC). Capabilities and functions provided by BMC 980 may vary considerably based on the type of information handling system. For example, the term baseboard management system may be used to describe an embedded processor included at a server, while an embedded controller may be found in a consumer-level device. As disclosed herein, BMC 980 represents a processing device different from processor 902, which provides various management functions for information handling system 900. For example, an embedded controller may be responsible for power management, cooling management, and the like. An embedded controller included at a data storage system may be referred to as a storage enclosure processor or a chassis processor.

System 900 may include additional processors that are configured to provide localized or specific control functions, such as a battery management controller. Bus 960 can include one or more busses, including a Serial Peripheral Interface (SPI) bus, an Inter-Integrated Circuit (I2C) bus, a system management bus (SMBUS), a power management bus (PMBUS), or the like. BMC 980 may be configured to provide out-of-band access to devices at information handling system 900. Out-of-band access in the context of the bus 960 may refer to operations performed prior to execution of firmware 972 by processor 902 to initialize operation of system 900.

Firmware 972 may include instructions executable by processor 902 to initialize and test the hardware components of system 900. For example, the instructions may cause the processor 902 to execute a power-on self-test (POST). The instructions may further cause the processor 902 to load a boot loader or an operating system (OS) from a mass storage device. Firmware 972 additionally may provide an abstraction layer for the hardware, such as a consistent way for application programs and operating systems to interact with the keyboard, display, and other input/output devices. When power is first applied to information handling system 900, the system may begin a sequence of initialization procedures, such as a boot procedure or a secure boot procedure. During the initialization sequence, also referred to as a boot sequence, components of system 900 may be configured and enabled for operation and device drivers may be installed. Device drivers may provide an interface through which other components of the system 900 can communicate with a corresponding device. The firmware 972 may include a basic input-output system (BIOS) and/or include a unified extensible firmware interface (UEFI). Firmware 972 may also include one or more firmware modules of the information handling system. Additionally, configuration settings for the firmware 972 and firmware of the information handling system 900 may be stored in the NVRAM 970. NVRAM 970 may, for example, be a non-volatile firmware memory of the information handling system 900 and may store a firmware memory map namespace 900 of the information handling system. NVRAM 970 may further store one or more container-specific firmware memory map namespaces for one or more containers concurrently executed by the information handling system.

Information handling system 900 may include additional components and additional busses, not shown for clarity. For example, system 900 may include multiple processor cores (either within processor 902 or separately coupled to the chipset 906 or through the PCIe buses 908), audio devices (such as may be coupled to the chipset 906 through one of the PCIe busses 908), or the like. While a particular arrangement of bus technologies and interconnections is illustrated for the purpose of example, one of skill will appreciate that the techniques disclosed herein are applicable to other system architectures. System 900 may include multiple processors and/or redundant bus controllers. In some embodiments, one or more components may be integrated together in an integrated circuit (IC), which is circuitry built on a common substrate. For example, portions of chipset 906 can be integrated within processor 902. Additional components of information handling system 900 may include one or more storage devices that may store machine-executable code, one or more communications ports for communicating with external devices, and various input and output (I/O) devices, such as a keyboard, a mouse, and a video display.

In some embodiments, processor 902 may include multiple processors, such as multiple processing cores for parallel processing by the information handling system 900. For example, the information handling system 900 may include a server comprising multiple processors for parallel processing. In some embodiments, the information handling system 900 may support virtual machine (VM) operation, with multiple virtualized instances of one or more operating systems executed in parallel by the information handling system 900. For example, resources, such as processors or processing cores of the information handling system may be assigned to multiple containerized instances of one or more operating systems of the information handling system 900 executed in parallel. A container may, for example, be a virtual machine executed by the information handling system 900 for execution of an instance of an operating system by the information handling system 900. Thus, for example, multiple users may remotely connect to the information handling system 900, such as in a cloud computing configuration, to utilize resources of the information handling system 900, such as memory, processors, and other hardware, firmware, and software capabilities of the information handling system 900. Parallel execution of multiple containers by the information handling system 900 may allow the information handling system 900 to execute tasks for multiple users in parallel secure virtual environments. For example, parallel execution of services described herein may occur in parallel virtualized containers.

The schematic flow diagram of FIG. 5 or flow charts of FIGS. 6 and 7 are generally set forth as logical flow chart diagrams. As such, the depicted order and labeled steps are indicative of aspects of the disclosed method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Machine learning models, as described herein, may include logistic regression techniques, linear discriminant analysis, linear regression analysis, artificial neural networks, machine learning classifier algorithms, or classification/regression trees in some embodiments. In various other embodiments, machine learning systems may employ Naive Bayes predictive modeling analysis of several varieties, learning vector quantization artificial neural network algorithms, or implementation of boosting algorithms such as Adaboost or stochastic gradient boosting systems for iteratively updating weighting to train a machine learning classifier to determine a relationship between an influencing attribute, such as received telemetry data, and a behavior change recommendation or action initiation and/or a degree to which such an influencing attribute affects the outcome of such a behavior change recommendation or action initiation.

If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. For example, although processors are described throughout the detailed description, aspects of the invention may be applied to the design of or implemented on different kinds of processors, such as graphics processing units (GPUs), central processing units (CPUs), and digital signal processors (DSPs). As another example, although processing of certain kinds of data may be described in example embodiments, other kinds or types of data may be processed through the methods and devices described above. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. An information handling system, comprising:
at least one processor; and
a memory coupled with the at least one processor and storing processor-readable code that, when executed by the at least one processor, is configured to perform operations including:
receiving telemetry information associated with a user performing a first user session of an application;
generating a health risk score of the user based on the telemetry information, the health risk score associated with a behavior of the user during the first user session; and
initiating, in response to the health risk score meeting a first criteria, a first action associated with the first user session.

2. The information handling system of claim 1, wherein the first action comprises pausing the first user session of the application for a predetermined amount of time.

3. The information handling system of claim 1, wherein the first action comprises lowering a volume of sound emitted from a first device.

4. The information handling system of claim 1, wherein the first action comprises transmitting a behavioral recommendation to a first device, the behavioral recommendation suggesting the user change the behavior.

5. The information handling system of claim 4, wherein:
the operations further include initiating, in response to the health risk score meeting a second criteria different than the first criteria, a second action associated with the first user session of the application, and
the second action comprises: terminating the first user session of the application at the first device; and initiating a second user session of the application at a second device.

6. The information handling system of claim 5, further comprising:
retrieving a mapping of a plurality of devices to a plurality of locations;
determining the second device for initiating the second user session based on the mapping of a plurality of devices to a plurality of locations and based on a first location for the first device being different from a second location for the second device.

7. The information handling system of claim 6, wherein determining the second device comprises determining a mobile device as the second device at the second location for initiating the second user session based on the first device being a stationary device.

8. The information handling system of claim 6, wherein determining the second device is further based on the telemetry information, and wherein determining the second device comprises determining, based on the telemetry information, that a head posture of the user associated with using the first device is different than a head posture of the user associated with using the second device.

9. The information handling system of claim 8, wherein determining the second device comprises determining the second device based on information that the second device is at least one of positioned at a different height relative to the user than the first device or positioned further away from the user than the first device.

10. A method comprising:
receiving, by an information handling system, telemetry information associated with a user performing a first user session of an application;
generating, by the information handling system, a health risk score of the user based on the telemetry information, the health risk score associated with a behavior of the user during the first user session; and
initiating, by the information handling system in response to the health risk score meeting a first criteria, a first action associated with the first user session.

11. The method of claim 10, further comprising:
generating a behavior score of the user based on the received telemetry information, the behavior score associated with the behavior of the user during the first user session; and
comparing the generated behavior score with a previously generated behavior score of the user, wherein the health risk score is generated in response to the behavior score being different than the previously generated behavior score.

12. The method of claim 10, wherein the application is a video game.

13. The method of claim 10, further comprising:
detecting active devices associated with the first user session;
retrieving a mapping of a plurality of devices to a plurality of locations, the plurality of devices including the active devices; and
determining a position of the user relative to the active devices based on the retrieved mapping.

14. The method of claim 10, further comprising:
retrieving a mapping of user furniture to a plurality of locations, and
wherein generating the health risk score of the user is further based on the retrieved mapping of user furniture.

15. The method of claim 10, wherein the first action comprises transmitting analytics to a first device, the analytics associated with the behavior of the user during the first user session.

16. A system comprising:
a first device associated with a first user session of an application, the first device configured to generate telemetry information associated with a user performing the first user session; and
a server configured to:
receive, from the first device, the telemetry information associated with the user performing the first user session of the application;
generate a health risk score of the user based on the telemetry information, the health risk score associated with a behavior of the user during the first user session; and
initiate, in response to the health risk score meeting a first criteria, a first action associated with the first user session.

17. The system of claim 16, further comprising a second device, and wherein the first action comprises migrating the first user session of the application from the first device to the second device.

18. The system of claim 17, wherein the server is further configured to determine the second device based on the telemetry information received from the first device, and wherein determining the second device comprises determining, based on the telemetry information, that a posture of the user associated with using the first device is different than a posture of the user associated with using the second device.

19. The system of claim 17, wherein the server is further configured to:
retrieve a mapping of user furniture to a plurality of locations;
determine a first seating apparatus associated with the first device based on the mapping; and
determine the second device based on the telemetry information received from the first device and a second seating apparatus associated with the second device based on the mapping.

20. The system of claim 16, wherein the application is a first application, and wherein the first action comprises: terminating the first user session of the application on the first device; and initiating a second user session of a second application different than the first application on the first device.
